(19) United States
(12) Patent Application Publication
(10) Pub. No.: US 2023/0181071 A1
(43) Pub. Date: Jun. 15, 2023

(54) BLOOD COLLECTION DEVICES, SYSTEMS, AND METHODS

(71) Applicant: Avia Vascular, LLC, Salt Lake City, UT (US)

(72) Inventors: Kevin Jerry Cook, Kaysville, UT (US); Jay Allen Muse, Salt Lake City, UT (US)

(73) Assignee: Avia Vascular, LLC, Salt Lake City, UT (US)

(21) Appl. No.: 17/988,606

(22) Filed: Nov. 16, 2022

Related U.S. Application Data
(63) Continuation of application No. PCT/US2021/032785, filed on May 17, 2021.
(60) Provisional application No. 63/026,048, filed on May 16, 2020.
(60) Provisional application No. 63/026,082, filed on May 17, 2020.

(51) Int. Cl. A61B 5/15; A61M 25/00; A61M 25/01

(52) U.S. Cl. CPC A61B 5/150992; A61M 25/0097; A61B 5/15003; A61M 25/0102

(57) ABSTRACT

A system can include a housing that can be coupled with a base catheter. The system can further include an access catheter of which a first portion is positioned within the housing and a second portion extends out of the housing through an opening when the access catheter is in a retracted position. The system can further include a stiffener encompassing at least a portion of the second portion of the access catheter, and the stiffener can be graspable at an exterior of the housing for application of force thereto to advance the access catheter from the retracted position to an extended position. When the housing is coupled to the base catheter, the first portion of the access catheter can be advanced through at least a portion of the base catheter as the access catheter is advanced to the extended position.

### CROSS-REFERENCE TO RELATED APPLICATIONS

This application is a continuation of International Application No. PCT/US2021/032785, filed May 17, 2021, titled BLOOD COLLECTION DEVICES, SYSTEMS, AND METHODS, which claims priority to U.S. Provisional Patent Application No. 63/026,048, filed May 16, 2020, titled BLOOD COLLECTION DEVICES, SYSTEMS, AND METHODS, and U.S. Provisional Patent Application No. 63/026,082, filed May 17, 2020, titled BLOOD COLLECTION DEVICES, SYSTEMS, AND METHODS; the entire contents of each of the foregoing applications are hereby incorporated by reference herein.

### TECHNICAL FIELD

Certain embodiments described herein relate generally to devices, systems, and methods for blood collection and further embodiments relate more particularly to devices, systems, and methods for facilitating blood collection via a previously placed catheter.

### BACKGROUND

Known devices, systems, and methods for drawing blood, including drawing blood using a previously placed catheter, suffer from a variety of drawbacks. Embodiments disclosed herein remedy, ameliorate, or avoid one or more of such drawbacks. Other or further uses and methods are also disclosed.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of an embodiment of a catheter system that can be used with a previously placed catheter system, such as a peripheral intravenous catheter system;

FIG. 2 is an exploded perspective view of the catheter system of FIG. 1;

FIG. 3 is a cross-sectional view of an intermediate region of an embodiment of a catheter portion of the catheter system of FIG. 1;

FIG. 4 is a perspective view of a proximal portion of the catheter system of FIG. 1,

FIG. 5 is a cross-sectional view of an intermediate region of the catheter system of FIG. 1, wherein the catheter system is in a fully retracted state;

FIG. 6A is a cross-sectional view of the catheter system of FIG. 1 in the fully retracted state being coupled to a proximal portion of an embodiment of a previously placed catheter system, which is shown in cross-section;

FIG. 6B is a cross-sectional view of a distal end of a catheter portion of the previously placed catheter system;

FIG. 7 is a cross-sectional view of the distal end of the previously placed catheter with a distal end of the catheter of FIG. 1 having been advanced past the distal tip of the previously placed catheter;

FIG. 8 is a cross-sectional view of intermediate and distal portions of another embodiment of a catheter system;

FIG. 9 is a cross-sectional view of intermediate and distal portions of another embodiment of a catheter system;

FIG. 10 is a perspective view of another embodiment of a catheter system;

FIG. 11A is a cross-sectional view of a distal tip of an embodiment of a catheter that is compatible with the catheter system of FIG. 10;

FIG. 11B is a cross-sectional view of a distal tip of another embodiment of a catheter that is compatible with the catheter system of FIG. 10;

FIG. 12 is a to plan view of another embodiment of a catheter system, wherein a portion of a housing is omitted to permit viewing of an interior of the housing;

FIG. 13 is a side elevation view of an embodiment of a housing that is compatible with the catheter system of FIG. 12;

FIG. 14 is a plan view of another embodiment of a catheter system, wherein a portion of a housing is omitted to permit viewing of an interior of the housing;

FIG. 15A is a cross-sectional view of an embodiment of a follow-on catheter that has been advanced through and coupled with a base catheter that has been pre-placed within a patient;

FIG. 15B is a cross-sectional view of a distal end of the coupled follow-on and base catheters within the vein of the patient;

FIG. 16 is a cross-sectional view of another embodiment of a follow-on catheter that includes a swelling layer on a tube;

FIG. 17 is a cross-sectional view of the follow-on catheter of FIG. 16 coupled with another embodiment of a base catheter, wherein the swelling layer has expanded to occlude an annular space between the follow-on catheter and the base catheter;

FIG. 18 is a cross-sectional view of another embodiment of a follow-on catheter that includes a swelling layer on a connector;

FIG. 19 is a cross-sectional view of another embodiment of a follow-on catheter that includes a sealing member on a hub;

FIG. 20 is a cross-sectional view of the follow-on catheter of FIG. 19 coupled with a base catheter, wherein the sealing member occludes an annular space between the follow-on catheter and the base catheter;

FIG. 20 is a cross-sectional view of another embodiment of a follow-on catheter that includes a distal section shaped as a male luer;

FIG. 21 is a cross-sectional view of the follow-on catheter of FIG. 20 coupled with a base catheter, wherein the male luer cooperates with a female luer shape of a hub of the base catheter to seal an annular region between the follow-on catheter and the base catheter;

FIG. 22 is a schematic cross-sectional view of another embodiment of a follow-on catheter that includes a male luer lock configuration;

FIG. 23 is a schematic cross-sectional view of another embodiment of a follow-on catheter that includes a freely rotating luer lock configuration; and

FIG. 24 depicts an embodiment of a follow-on catheter.

### DETAILED DESCRIPTION

Embodiments of the present disclosure relate generally to devices, systems, and methods for blood collection (also referred to as blood draws, blood aspiration, phlebotomy procedures, etc.). The blood collection can be achieved via a cannula system that is inserted into a previously placed catheter, such as a peripheral intravenous (PIV) catheter. In certain embodiments, the cannula system can provide a desirable alternative to venipuncture.

In one context, fluid, particularly blood, is drawn from patients on a routine basis in many hospitals, clinics, and laboratories. One of the most common ways to draw blood is venipuncture, which is a method that involves inserting a needle through the skin and into an underlying vein. In some instances, blood can be drawn as frequently as every six hours. Further, patients can be subjected to multiple attempts each time a needle is inserted into the skin, and the more frequent the withdrawals become, the more difficult it can become to find a location for the next withdrawal. Each attempt can be painful and a nuisance. Other options for the withdrawal of blood and other fluids, however, are limited, and can often be even more painful than venipuncture. Some of these options include the use of peripherally inserted central catheters (PICC lines), central lines, repeated peripheral venipuncture, and groin sticks.

In some instances, blood may be drawn via a peripheral intravenous catheter at the time of insertion. In many instances, however, clots or fibrin sheaths can form at the tip of an intravenous catheter over time, so it is generally not desirable to draw blood from peripheral intravenous catheters, as previously designed, at any significant time after placement. In various instances, the obstructions that form at the distal tip can fully obstruct the distal tip, thereby entirely preventing withdrawal of blood through the catheter. In other instances, the obstructions may only partially obstruct the distal tip, but may affect fluid flow through the catheter in such a way as to promote hemolysis and/or otherwise reduce the quality of the blood withdrawn through the catheter and/or reduce a speed of the withdrawal.

In some instances, the intravenous catheter can lack sufficient rigidity to remain fully patient during a blood draw. For example, in some instances, the catheter is formed of a material that softens over time when within the patient vasculature. While blood draws may be possible upon initial placement of the catheter, blood draws may become increasingly difficult and ultimately impossible after the initial placement as the catheter softens. The catheter lumen may collapse when negative pressure applied at a proximal end of the catheter for an attempted blood draw, thereby inhibiting or preventing blood withdrawal.

In certain instances, even when blood is able to be withdrawn through a softened catheter, the quality of the drawn blood, relative to the quality of blood drawn immediately after placement of the catheter, may deteriorate as the catheter softens. Without being bound by theory, this quality reduction may be due to the lumen defined by the catheter becoming more tortuous or otherwise changing shape as the catheter softens. For example, upon initial placement, the catheter may have relatively few curves and/or regions of the catheter that extend through the skin and the vessel wall may define a rounded cross-sectional profile. Over time, the softening or softened catheter may become compliant so as to conform to tortuous anatomy through which it passes and/or the cross-sectional profile at the insertion regions may flatten or otherwise change shape. Blood drawn through these altered regions may, for example, be more susceptible to hemolysis.

In still other or further instances, an opening at the catheter tip may suction against a feature of the vessel anatomy, such as a valve or the vessel wall, during an attempted blood draw, thereby preventing blood withdrawal through the opening.

For one or more of the foregoing reasons, and/or for other reasons, it can be desirable to provide a conduit or cannula through a previously placed catheter (e.g., a PIV catheter) to achieve a high-quality blood draw through the catheter. In some instances, the cannula can have sufficient rigidity to provide a patient lumen through which blood can readily pass. In other or further instances, the cannula can straighten or otherwise reduce a tortuousness of a path along which the drawn blood passes, which may yield a laminar flow of blood through the cannula. In other or further instances, the distal end of the cannula may be placed distally relative to the tip of the previously placed catheter, which may, for example, avoid interference from clots or fibrin sheaths; permit access to a region beyond an otherwise obstructing anatomical feature, such as by moving past one or more venous valves and/or away from a vessel wall; permit movement to a region of increased blood flow; and/or move away from a region of vascular trauma due to venipuncture and catheter insertion, any or all of which can result in substantially improved blood draws, such as, for example, by establishing blood flow and/or by reducing hemolysis of the sampled blood.

Certain embodiments disclosed herein can remedy, ameliorate, or avoid one or more limitations or drawbacks of known systems in which a catheter is inserted through a previously placed catheter for purposes of blood collection. One or more of these and/or other advantages will be apparent from the present disclosure.

In some instances, a catheter is introduced into a patient, and subsequently, a cannula is introduced into the patient through the catheter. The catheter may be referred to herein as a placed catheter, a preplaced catheter, an anchor catheter, or a base catheter. The cannula may, in some instances, also be referred to as a fluid channeler, fluid extraction member, catheter, etc. In many instances, the cannula will have properties different from the placed catheter. For example, in some instances, the cannula may be stiffer or more rigid than the placed catheter. The cannula may, in some instances, define an open passageway through which blood can pass from the vessel and out of the patient through the cannula into any suitable blood collection device. The cannula may, in some instances, straighten or otherwise reduce a tortuous path of a lumen defined by the catheter. In some instances, the cannula may be advanced past a distal end of the placed catheter.

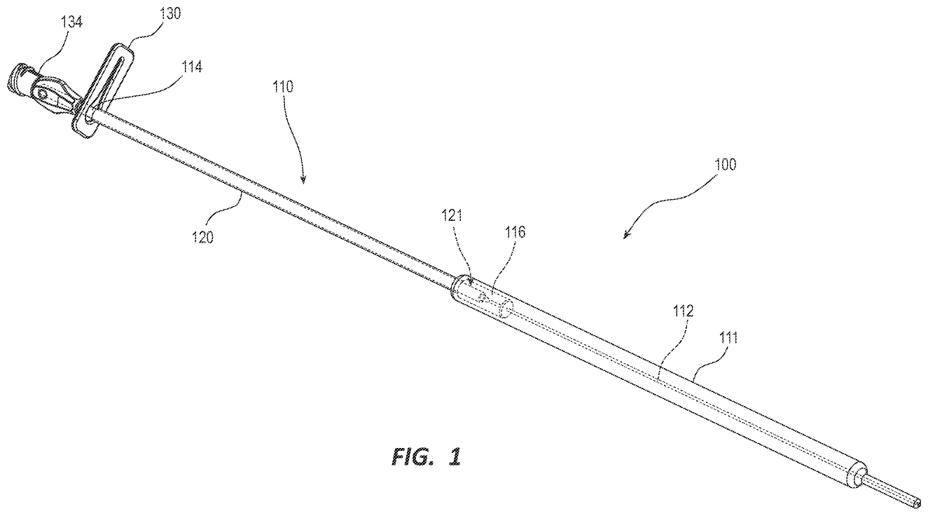

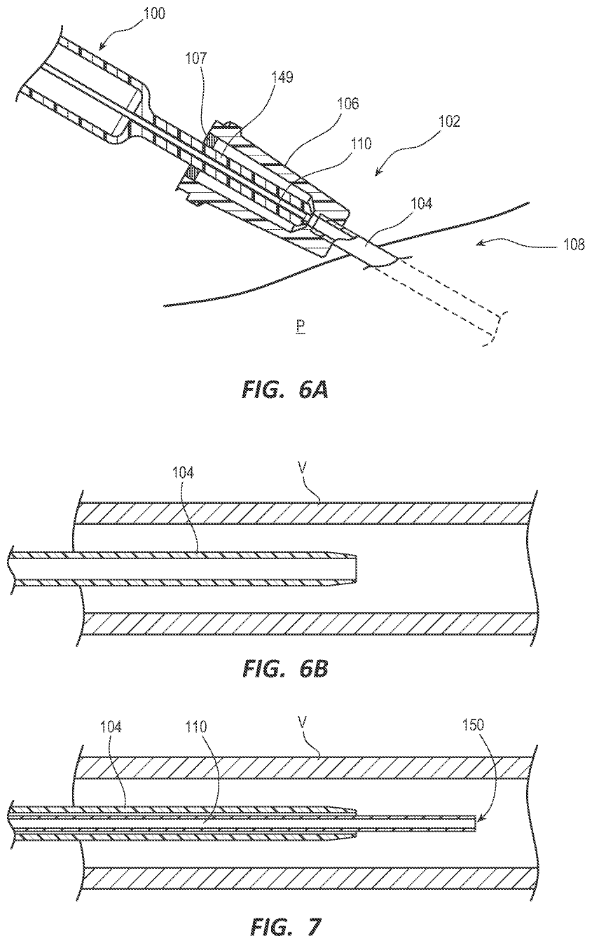

FIG. 1 depicts an embodiment of a catheter system 100. The catheter system 100 can be configured for use with a previously placed catheter system, such as the illustrative peripheral intravenous catheter system 102 depicted in FIGS. 6A and 6B, which includes a catheter 104 and a hub 106. The pre-placed peripheral intravenous catheter system 102 can be inserted into a patient such that at least a distal end of the catheter 104 extends into a vessel V (e.g., a vein) of the patient (FIG. 6B) and such that a proximal portion of the system 102, including the hub 106, is accessible at an exterior of the patient.

With reference to FIGS. 1, 6A, 6B, and 7, and as further discussed hereafter, the catheter system 100 can include a cannula or catheter 110 of which a portion can be inserted through the previously placed catheter system 102. The catheter 110 of the system 100 (which may also be referred to as an access catheter or system catheter) can be moved from a retracted position (FIG. 6A) and advanced through the catheter 104 of the pre-placed system 102 to a deployed position (FIG. 7) such that a distal tip of the catheter 110 extends beyond a distal tip of the catheter 104. The catheter 110 may be said to bypass or supersede the catheter 104 and can be used for functions that might otherwise have been performed directly via the catheter 104, such as infusions and/or blood collections. Accordingly, the catheter system 100 may also be referred to herein as an access, bypass, superseding, follow-on, add-on, or transient system, whereas the catheter system 102 may be referred to as a pre-placed, placed, base, or anchor system.

In some embodiments, the catheter system 100 can be devoid of any needles or other piercing members configured to readily provide access through the skin of a patient. Whereas a pre-placed system 102 may include such a piercing member for initial placement of the catheter 104 within the patient vasculature, no such piercing member is needed for a follow-on system 100 to introduce the catheter 110 into the patient vasculature.

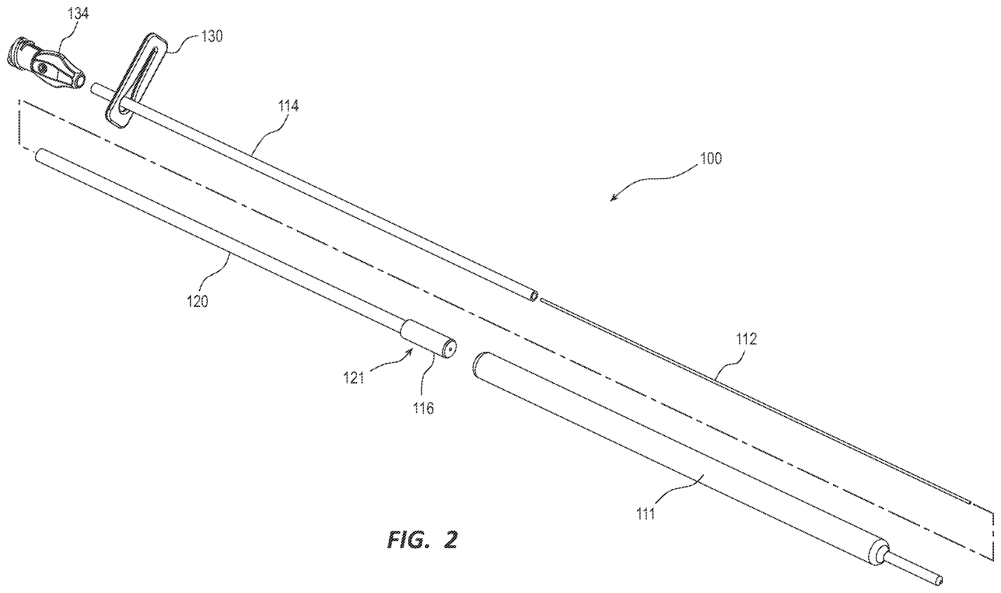

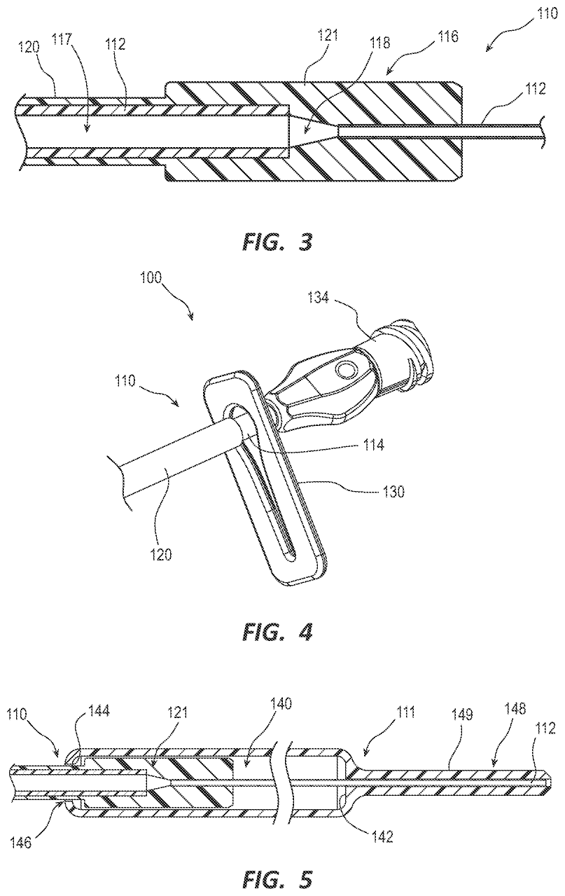

With reference to FIGS. 1 and 2, the catheter 110 can be configured to move relative to a housing 111, which may also be referred to as a sheath or distal hub. Stated otherwise, at least a portion of the catheter 110 can be positioned within the housing 111, and the catheter 110 can translate through the housing 111. The catheter 110 can include a distal portion 112 and a proximal portion 114. The distal portion 112 may also be referred to as a distal tube or distal catheter section, and the proximal portion 114 may also be referred to as a proximal tube, proximal catheter section, or proximal extension. In some embodiments, an inner and outer diameter of the proximal portion 114 are larger than an inner and outer diameter of the distal portion 112. For example, a lumen 117 (see FIG. 3) of the catheter 110 within the proximal portion 114 can expand to a larger diameter in more proximal portions of the catheter 110.

With reference to FIGS. 2 and 3, in some embodiments, the catheter 110 incudes a junction 116 that joins the distal and proximal portions 112, 114 together. Any suitable junction 116 is contemplated. In the illustrated embodiment, the junction 116 is overmolded onto the distal and proximal portions 112, 114. In other embodiments, the junction 116 may be molded separately, and the distal and proximal portions 112, 114 can be bonded thereto. The junction 116 can include an internal transition region 118 at which the inner lumen 117 of the catheter 116 increases in size from the inner diameter of the distal portion 112 to the inner diameter of the proximal portion 114.

In some embodiments, the junction 116 can include a follower 121 that can be moved passively as the catheter 110 is advanced or retracted. In the illustrated embodiment, the follower 121 comprises a substantially tubular region of increased diameter, relative to other portions of the catheter 110. The follower 121 can, in some instances, interface with the housing 111 (e.g., contact the housing 111 at an interior of the housing) during advancement and/or retraction of the catheter 110. The follower 121 may also be referred to as a stopper. For example, in the illustrated embodiment, the junction 116, acting as the follower 121, has a raised profile such that an outer surface of the junction 116 can interface with an interior of the housing 111. In some embodiments, an outer geometry of the raised junction 116 is configured to slide or glide within an inner geometry of the housing 111. Interfacing arrangements other than simple sliding are also contemplated, as further discussed below. Proximal and distal ends of the follower (or stopper) 121 can abut internal surfaces at the proximal and distal ends of the housing 111, respectively, to retain the follower 121 within the housing 111. In other embodiments, the follower 121 and the junction 116 may be separate components and/or may be positioned at different longitudinal locations along the catheter 110.

With reference to FIGS. 1-3, in some embodiments, the catheter 110 further includes a stiffener 120, which may alternatively be referred to as a stiffening sheath. In the illustrated embodiment, the stiffener 120 is substantially tubular and extends along a substantial majority of a length of the proximal catheter portion 114. For example, in various embodiments, the stiffener 120 can encompass or otherwise cover no less than 50, 60, 70, 80, or 90 percent of a portion of the proximal catheter portion 114 that extends proximally from or, stated otherwise, that is external to the housing 111 when the catheter 110 is in a retracted position.

The stiffener 120 portion of the catheter 110 may be relatively rigid to facilitate advancement and/or retraction of the catheter 110 relative to the housing 111. For example, a hardness of the stiffener 120 can be greater than a hardness of the proximal catheter portion 114. In some embodiments, the stiffener 120 is attached to the proximal catheter portion 114 along at least a portion of a length of the stiffener 120 that covers the proximal catheter portion 114. Distally directed forces on the stiffener 120 can cause the stiffener 120 and the proximal catheter portion 114 to advance distally through the housing 111 in unison.

The stiffener 120 can initially extend proximally through an opening at a proximal end of the housing 111 to be gripped by a user. Stated otherwise, the stiffener 120 can be graspable at an exterior of the housing 111. The user can advance or retract the catheter 110 relative to the housing 111 via the stiffener 120. For example, a user can grip the stiffener 120 with one hand and grip the housing 111 with another hand. The user can thereby urge the stiffener 120 distally. In other or further instances, a user can advance and/or retract the proximal catheter portion 114 by gripping an exposed proximal end of the proximal catheter portion 114 (e.g., a region not covered by the stiffener 120) and/or a connector 134 attached to the proximal catheter portion 114, whether separately or in conjunction with gripping the stiffener 120.

In some embodiments, the stiffener 120 is connected with the junction 116. For example, in the illustrated embodiment, the stiffener 120 and the junction 116 are formed as a unitary piece of material, which may be overmolded onto an external surface of a distal region of the proximal catheter portion 114 and the proximal end of the distal catheter portion 112. In other embodiments, the stiffener 120 and the junction 116 may be separate pieces, which may or may not be joined together. The stiffener 120 and/or the junction 116 may be formed in any other suitable manner.

With reference to FIGS. 1 and 4, in some embodiments, a proximal end of the stiffener 120 terminates at a position that is distal to a proximal end of the proximal catheter portion 114. Stated otherwise, a proximal end of the proximal catheter portion 114 may be left exposed, or uncovered relative to the stiffener 120, which can permit or facilitate selective opening and closing of the proximal catheter portion 114, such as to prevent egress of blood, via a clamp 130 of any suitable variety. In the illustrated embodiment, the clamp 130 is a standard sliding clamp.

In other embodiments, the stiffener 120 and/or the junction 116 may be omitted. For example, in some embodiments, the distal and proximal portions 112, 114 of the catheter 110 may be joined in other manners. In other embodiments, the distal and proximal portions 112, 114 may be formed of a unitary catheter body, and in still further embodiments, the distal and proximal portions 112, 114 may comprise different portions of a substantially uniform material or arrangement (e.g., a unitary tube of substantially constant properties and dimensions) that extends between the distal and proximal tips of the catheter 110. In some embodiments, the material properties of the catheter 110 may change in the longitudinal direction. For example, in some embodiments, a durometer of the catheter 110 may be larger in the proximal portion 114 than in the distal portion 112. In some embodiments, at least the proximal portion 114 includes braided and/or coiled wires that provided added stiffness.

With reference to FIGS. 1, 2, and 4, in some embodiments, the catheter assembly 100 includes the connector 134, which can be a fitting of any suitable variety, at the proximal end of the catheter 110. The connector 134 can be configured to couple with any suitable fluid source and/or fluid collection device or may, in other embodiments, be integrally formed with a fluid source and/or a fluid collection apparatus. For example, in the illustrated embodiment, the connector 134 comprises a female luer connector configured to couple with a syringe, which may be used for infusion or blood collection. The illustrated connector 134 and/or other varieties of connectors may be used for connection to blood draw apparatus, such as evacuated blood collection tubes (e.g., Vacutainer® tubes available from Becton Dickinson) and/or apparatus therefor. For example, in some embodiments, the connector 134 may include or may be configured to connect with a Vacutainer® one-use holder, via which blood draws may be made into one or more Vacutainer® tubes.

In the illustrated embodiment, the connector 134 does not provide any valving. Instead, the catheter system 100 includes a separate clamp 130 by which blood flow may be selectively stopped and started. In other embodiments, the connector 134 may comprise a valve and/or the clamp 130 may be omitted. For example, in some embodiments, the connector 134 may comprise a needleless connector valve of any suitable variety. In certain of such embodiments, the stiffener 120 may extend fully to the connector 134. For example, the stiffener 120 and/or the connector 134 may fully cover at least a proximal end of the proximal catheter portion 114.

With reference to FIG. 5, in the illustrated embodiment, the housing 111 is substantially tubular or cylindrical and defines an internal cavity 140 through which the follower 121 can translate. An internal surface of the housing 111 can be conducive to translation and/or rotation of the follower 121, such as by being in a sliding relationship therewith. In other embodiments, the follower 121 may be constrained to translational motion without rotation, such as by way of any suitable keying between the housing 111 and the follower 121. For example, an internal surface of the housing 111 may have a geometry that includes one or more flat surfaces, and an outer surface of the follower 121 can have a complementary geometry with one or more corresponding flat surfaces. Interaction of, e.g., the flat surfaces can resist rotation of the follower 121 relative to the housing 111. Further examples of interfaces between the housing 111 and the follower 121 are discussed below with respect to other embodiments.

The housing 111 can include a distal stop 142 and a proximal stop 144, which can delimit forward and rearward movement, respectively, of the follower 121 relative to the housing 111. The follower 121 can be constrained by the stops 142, 144 to remain within the housing 111 to prevent removal of the access catheter 110 from the housing 111. The proximal end of the housing 111 can include an opening 146 through which a proximal portion of the catheter 110 can pass.

The housing 111 can include an interface 148 at a distal end thereof for interfacing or coupling with the placed catheter system 102. In some instances, the interface 148 includes a securement mechanism to fixedly secure the housing 111 to the placed catheter system 102. Any suitable securement mechanism (e.g., threading, such as may be used in luer lock systems) is contemplated, although none are depicted in the drawings with respect to the system 100.

With reference to FIG. 6A, in some embodiments, the interface 148 comprises an elongate extension 149, which can be inserted into the hub 106. In some embodiments, the extension 149 can be configured to open, bypass, defeat, extend through, or otherwise provide passage for the catheter 110 through a seal 107 of the hub 106. The seal 107 is shown schematically in FIG. 6A. The hub 106 may be attached to or otherwise include (e.g., be integrally formed as) any of a variety of needleless connector valve configurations. Such configurations can include a seal 107 that generally serves to prevent egress of blood from the hub 106, but which can selectively be opened for infusion and/or aspiration.

In other or further embodiments, the extension 149 may be configured to couple with the hub 106 so as to form a seal therewith that is independent of a separate seal 107. For example, in some embodiments, the hub 106 may be devoid of a seal 107 and may instead define a luer interface. For example, the hub 106 may be formed as a female luer connector or as a female luer lock connector. In some embodiments, the extension 149, or at least a distal end thereof, can be shaped as a male luer for insertion into and frictional engagement with the hub 106. In other embodiments, the interface 148 may comprise a luer lock connection, such that the system 100, e.g., in its entirety, or an internally threaded sleeve may be rotated relative to the system 102 to securely connect the systems 100, 102 to each other, and may be rotated again after use for disconnection. Any other suitable interfacing and/or locking arrangement between the systems 100, 102 is contemplated.

The various components of the system 102 can be formed in any suitable manner and of any suitable material, as understood from the functions of those components. In some embodiments, the housing 111 may be formed of a rigid thermoplastic, such as, for example, polycarbonate or polycarbonate-ABS. The material may be selected for its strength and/or low-friction properties.

In some embodiments, the distal catheter portion 112 may be formed of, for example, polyurethane, polyimide, nylon, or PEEK and may include one or more lubricious coatings, such as silicone or a different hydrophilic coating. The material or materials may be selected for such properties as dimensional stability and lubricity.

In some embodiments, the proximal catheter portion 114 may be formed of, for example, polyurethane, Pebax, or a flexible transparent or translucent thermoplastic. The material may be selected for such properties as transparency or translucence, which can facilitate viewing of blood flow therethrough, and/or flexibility.

In some embodiments, the junction 116 may be formed of, for example, polyurethane, polycarbonate, polycarbonate-ABS, acetal, nylon, PEEK, or a rigid thermoplastic. In some embodiments, the material may desirably have a good bond strength with the materials of each of the proximal and distal catheter portions 114, 112, may be suitable for overmolding, and/or may exhibit relatively low friction. In some embodiments, the junction 116 and the stiffener 120 are formed of the same material, and in further embodiments, both may be overmolded to the catheter portions 114, 112. In other instances, the junction 116 and the stiffener 120 may be formed of different materials. The stiffener 120 may be formed via processes other than overmolding, and may be attached to the junction 116 (such as via overmolding, adhesive bonding, etc.) and/or the proximal catheter portion 114.

Illustrative examples of using the catheter system 100 will now be described. In certain embodiments, the catheter system 102 will be pre-placed within the vein of a patient according to accepted protocols. The catheter system 102, for example, may be a peripherally intravenous (PIV) catheter.

The catheter system 100 may be provided with the catheter 110 in a fully retracted state, configuration, or orientation, such as that depicted in FIG. 1. The full system 100 may be provide in a sterile state. The system 100 may preserve the sterility of all components or portions thereof that remain within the housing 111. For example, the follower 121 may remain entirely within the housing 111 throughout use such that the sterility thereof may be preserved. Similarly, the distal catheter portion 112 may be maintained within the housing 111 in a sterile state and then pass directly into the pre-placed catheter system 102.

With the catheter system 100 in its initial, preset, pre-packaged, or retracted state, the catheter system 100 may be coupled to the pre-placed catheter system 102 in any suitable manner. In some instances, the user ensures that the clamp 130 is in a closed configuration prior to such coupling. In the illustrated embodiment, the extension 149 of the housing 111 can be advanced through the seal 107 of the hub 106 of the catheter system 102 to provide access to a lumen of the catheter 104 of the catheter system 102.

The user can grip the housing 111 with one hand and grip the stiffener 120 with the other. As previously discussed, in other or further instances, the user can grip an exposed proximal end of the proximal catheter portion 114 and/or the connector 134 instead of or in addition to the stiffener 12. Gripping a proximal portion of the system 100 in this manner, the user can advance the stiffener 120, proximal catheter portion 114, and/or the connector 134 distally relative to the housing 111 to advance the distal catheter portion 112 into and through the catheter 104 of the pre-placed catheter system 102. The user can selectively advance the distal tip of the distal catheter portion 112 past a distal tip of the pre-placed catheter 104 and into the blood stream of the vessel V, as shown in FIG. 7. In various instances, the distal tip of the distal catheter portion 112 is advanced past the distal tip of the pre-placed catheter 104 by a sufficient distance and/or to otherwise achieve an orientation in which one or more openings 150 of the distal catheter portion 112 is unobstructed by clots or fibrin sheaths, is extended past one or more venous valves, is positioned away from a vessel wall, and/or is positioned within a region of increased blood flow, any or all of which can result in substantially improved blood draws. In the illustrated embodiment, the opening 150 is a simple circular and planar opening oriented transversely relative to a longitudinal axis of the distal catheter portion 112 and positioned at the distal tip of the catheter portion 112. As further discussed below, any other suitable variety of opening configuration is contemplated.

In the case of blood collection, a blood collection device, such as any of those described above, can be coupled to the connector 134. In the case of infusion, an infusion device, such as any of those described above, can be coupled to the fitting. In either case, the clamp 130 can be moved to an open configuration prior to the blood draw or infusion to permit passage of fluid through the catheter 110.

After the withdrawal or delivery of fluid via the catheter 110, the clamp 130 may again be closed. The stiffener 120 may be gripped and moved proximally relative to the housing 111 to draw the distal end of the catheter 110 into the housing 111. The catheter system 100 can be decoupled from the catheter system 102 and appropriately disposed of.

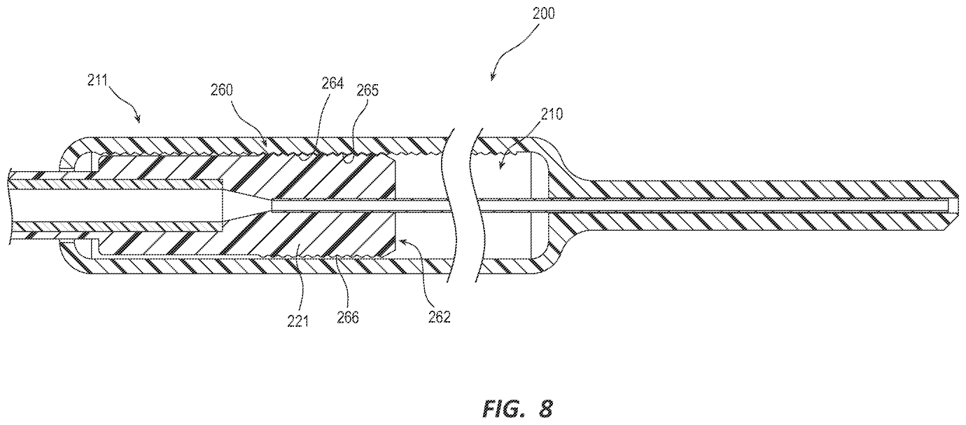

FIG. 8 depicts another embodiment of a catheter system 200 that can resemble the catheter system 100 discussed above in many respects. Accordingly, like features are designated with like reference numerals, with the leading digits incremented to “2.” Relevant disclosure set forth above regarding similarly identified features thus may not be repeated hereafter. Moreover, specific features of the catheter system 200 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the catheter system 200. Any suitable combination of the features and variations of the same described with respect to the catheter systems discussed above can be employed with the catheter system 200, and vice versa. This pattern of disclosure, and the contemplated combination of any of the described features, applies equally to further embodiments depicted in subsequent figures and described hereafter, wherein the leading digits may be further incremented.

The catheter system 200 includes a housing 211 and a catheter 210 that resemble similarly numbered features in many respects. The catheter system 200 can further include a proximal hub and a clamp similar to the proximal hub 134 and the clamp 130 described above, although not shown in FIG. 8.

The housing 211 can include a positioning member 260 of any suitable variety. The positioning member may also be referred to as a resistance member, adjustment member, depth-control member, etc. The positioning member 260 can be configured to interact with a follower 221 of the catheter 210 to permit controlled movement of the catheter 210 relative to the housing 211. In particular, the follower 221 may likewise include a positioning member 262 that interfaces with the positioning member 260 of the housing 211 to achieve the controlled movement. Interaction of the positioning members 260, 262 can assist in maintaining a fixed relationship (e.g., longitudinal relationship) between the housing 211 and the catheter 210 after advancement of the catheter 210 to a desired deployment orientation.

In the illustrated embodiment, the positioning member 260 of the housing 211 is formed as a longitudinal track 264 that includes a series of teeth 265. For example, the teeth 265 can be formed as one or more transversely oriented ribs. The track 264 extends along substantially a full length of the tubular region of the housing 211 within which the follower 221 resides. In other embodiments, the track 264 can extend along no less than 50, 60 70, 80, or 90 percent of a length of the tubular region of the housing 211. In the illustrated embodiment, the track 264 is positioned longitudinally along a single side of the housing 211. Other arrangements of the track 264 are contemplated.

The positioning member 262 of the follower 221 can include a series of adjacent protrusions or ribs 266 that are configured to be received within the recesses between adjacent teeth 265 of the track 264. The size and pitch of the teeth 265 and the ribs 266 can control the sensitivity of the positioning system. For example, a relatively larger number of teeth 265 over a given length can yield a relatively larger number of stable positioning configurations between the catheter 210 and the housing 211, or stated otherwise, can permit a distal end of the catheter 210 to extend past a distal end of a pre-placed catheter by any of a relatively larger number of fixed lengths. The positioning members 260, 262 can provide the user with tactile feedback regarding a speed and/or an amount of advancement of the catheter 210 relative to the housing 211.

Other suitable arrangements of the positioning members 260, 262 are contemplated. Moreover, in other or further embodiments, friction-enhancing treatments may be provided on one or more of and/or between the follower 221 and the sidewall of the housing 211. For example, in some embodiments, one or more surfaces may be roughened and/or friction grease may be applied between surfaces of the follower 221 and the housing 211.

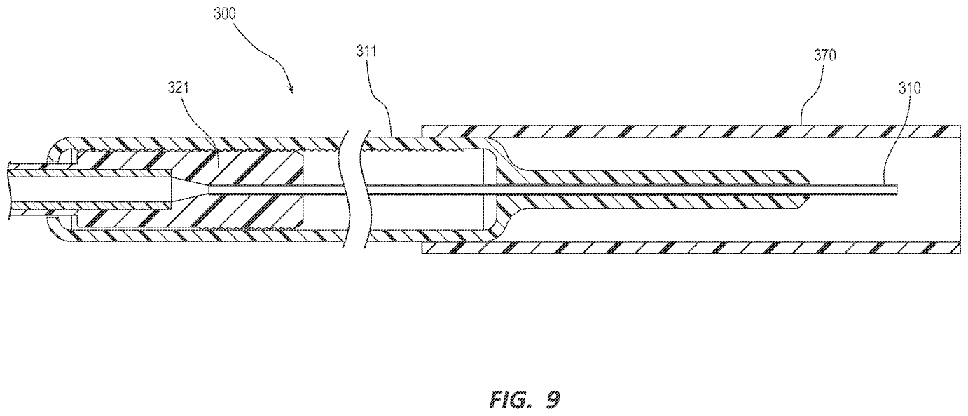

FIG. 9 depicts another embodiment of a catheter system 300 that can resemble the catheter systems 100, 200 discussed above in many respects and, as previously described, features of these various systems may be incorporated into each other in any suitable manner. The catheter system 300 is configured such that a distal end of a catheter 310 extends past a distal end of a housing 311 when the catheter 310 is in a fully retracted state. For example, as depicted, the catheter 310 can include a follower 321 of which a proximal end abuts a proximal end of the housing 311 when the catheter 310 is in the fully retracted state, such that no further proximal movement of the catheter 310 is possible relative to the housing 311. In this fully retracted state, a distal portion of the catheter 310 extends through the distal end of the housing 311, and a distal tip of the catheter 310 is exposed or, stated otherwise, is positioned external to the housing 311. In other embodiments, the distal tip of the catheter 310 can be positioned within the housing 311 when in a retracted state (such as in other embodiments previously described an shown in, e.g., FIGS. 1, 5, and 8).

In some embodiments, the catheter system 300 can include a cap 370, which may also be referred to as a sterility cap. The cap 370 can prevent inadvertent or undesirable contact with the distal end of the catheter 310 prior to use, particularly where the distal tip of the catheter 310 is exposed when in the retracted state. This can prevent damage to the tip of the catheter 310 and/or can prevent contamination of the tip prior to use. The cap 370 can be removably coupled to the housing 311 in any suitable manner. The catheter 310 can be packaged and sterilized with the cap 370 in place, and the cap 370 can be removed just prior to use of the catheter system 300.

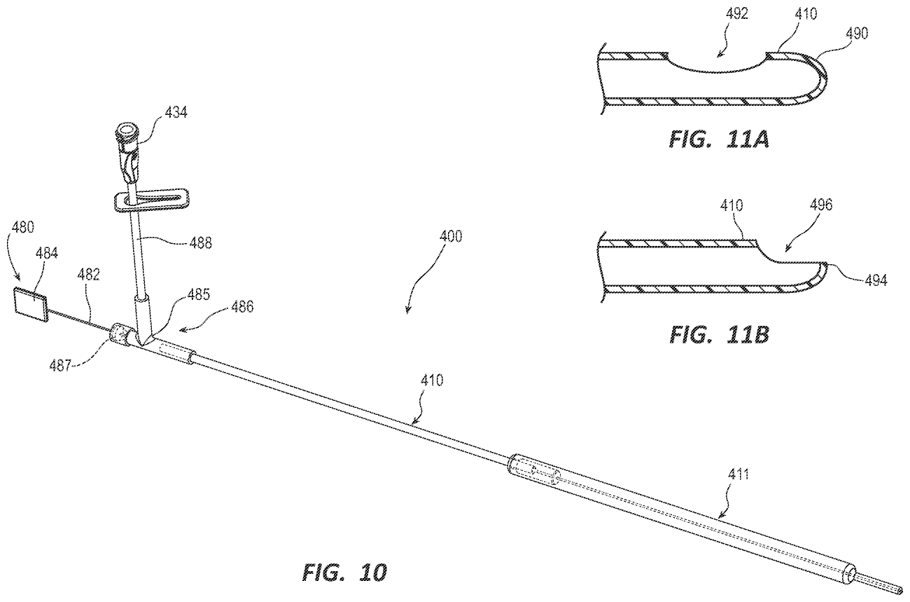

FIG. 10 depicts another embodiment of a catheter system 400 that can resemble the catheter systems 100, 200, 300 discussed above in many respects and, as previously described, features of these various systems may be incorporated into each other in any suitable manner. The system 400 can include a housing 411 and a catheter 410, each of which can be configured similarly or identically to any of the similarly named and numbered components previously described. As an exception, rather than being directly attached to a proximal hub, a proximal end of the catheter 410 can instead be directly coupled to a T-shaped hub 485 of a T-lock extension set or T-lock assembly 486. Other configurations than T-shaped are contemplated, such as, e.g., Y-shaped configurations. The T-lock assembly 486 can include an extension tube or fluid path extension 488 that includes a proximal hub 434 attached to a proximal end thereof. The assembly 486 can further include a septum, seal, or valve 487 of any suitable variety through which a portion of an obturator assembly 480 can be inserted and/or removed.

The obturator assembly 480 can include an internal stiffening member, stylet, or obturator 482 and a grip or handle 484. In some embodiments, the system 400 may be provided in an initial, pre-packaged, or read-to-use configuration in which the obturator 482 is pre-inserted through the hub 485 and extends through the catheter 410 to a distal tip thereof. The handle 484 can be used to facilitate removal of the obturator 482 from the catheter 410. For example, the handle 484 can be sized and shaped to be readily gripped for removal.

The obturator 482 can be formed of any suitable material and can be relatively stiffer than at least a distal portion of the catheter 410 to assist in advancing the catheter 410 through the vasculature, but can be sufficiently flexible to deflect laterally to follow tortuous paths through the vasculature, in certain instances. In some instances, use of an obturator 482 can enable or facilitate use of a thin-walled catheter 410, which can improve the quality of blood draws, such as by reducing hemolysis and/or increasing blood flow for quicker draws. The obturator 410 can be resiliently or elastically flexible to naturally return from deflection to an unstressed configuration (e.g., a substantially rectilinear configuration). In various embodiments, the obturator comprises one or more of stainless steel, Nitinol, polypropylene, PEEK, or polyurethane.

In some embodiments the obturator 482 includes a coating, such as silicone or a hydrophilic coating. The coating can assist with removal of the obturator 482 from the catheter 410. In some instances, the coating may be highly frictional relative to the material of the catheter 410, which can assist in maintaining the obturator 482 coupled with the catheter 410 during insertion. Once removal of the obturator 482 is desired, saline can be injected through the proximal hub 434 of the T-lock assembly 486 to activate or lubricate the coating to enable or facilitate removal of the obturator 482 from the catheter 410.

The valve 487 can form a fluid tight seal around the obturator 482 when the obturator 482 extends therethrough. In further embodiments, the valve 487 can be configured to self-seal to prevent fluid egress from the hub 485 upon removal of the obturator 482. For example, in some embodiments, the valve 487 can comprise an elastomeric septum with one or more layers that each include one or more slits therethrough. Any suitable arrangement of the valve 487 is contemplated.

In some embodiments, the distal end of the catheter 410 is substantially open, such as the open end of the catheter 110 depicted in FIG. 7. In such embodiments, a distal tip of the obturator 482 may extend distally past the tip of the catheter 410, may be flush therewith, or may be proximally retracted relative thereto. In other embodiments, at least a portion of the catheter 410 at a distal end thereof functions as a catch or stop against which the obturator 482 may press when the obturator 482 is advanced distally.

For example, with reference to FIG. 11A, in some embodiments, a distal end of the catheter 410 includes a closed distal tip 490 and one or more side ports 492 that may be formed in any suitable manner (e.g., skiving). The distal tip of the obturator 482 may abut against an internal surface of the closed distal tip 490 to assist in advancing the catheter 410 through the vasculature. With reference to FIG. 11B, in some embodiments, the distal end of the catheter 410 includes a partially covered distal tip 494 and an end port 496. The partially covered distal tip 494 can sufficiently interfere with the distal end of the obturator 482 to prevent the obturator 482 from moving distally past the distal tip 494. Stated otherwise, the tip 494 can include a ridge that covers the opening and the obturator 482 can abut the ridge of the tip 494 and push it distally, which in turn pulls the catheter 410 distally through the vasculature.

With reference again to FIG. 10, embodiments of the system 400 can be used in a variety of manners. As previously noted, in some instances, the obturator 482 may be preinstalled within the catheter 410. In other instances, a user may initially advance the obturator 482 into the catheter 410.

The housing 411 can be coupled to a pre-placed catheter system (e.g., the catheter system 102 discussed above) in any suitable manner, such as those previously discussed. In some instances, a user will hold the housing 411 steady (e.g., substantially fixed relative to the catheter system 102) with one hand and with the other hand, will grasp one or more of the exposed proximal portion of the catheter 410, the T-lock assembly 486, or the obturator assembly 480 to advance the catheter 410 forward relative to the housing 411 to move the catheter 410 past a distal tip of the pre-placed catheter. In various instances, a user may grip and advance forward only the exposed proximal end of the catheter 410, only the T-shaped hub 485 and/or the extension tube 488, or only the obturator assembly 480.

The obturator 482 can stiffen the catheter 410, which can help with advancing the distal end of the catheter 410 through the pre-placed catheter and/or through vasculature features beyond a distal end of the pre-placed catheter. For example, the obturator 482 can assist in moving the catheter 410 past venous valves.

Once the catheter 410 has been moved to a desired position, the obturator assembly 480 may be removed. In some instances, prior to removal, a syringe or other fluid delivery device is coupled to the proximal hub 434 and saline is delivered through the proximal hub 434, the extension tube 488, the intermediate hub 485, and the catheter 410, which can activate and/or lubricate the obturator to enable or facilitate removal. The user may grip the hub 486 with one hand and pull proximally on the handle 484 of the obturator assembly 480 with the other until the obturator 482 is fully removed. The valve 487 can automatically seal upon removal of the obturator 482. Blood draws and/or infusions can take place via the proximal hub 484 in manners such as previously disclosed.

With continued reference to FIG. 10, in other embodiments, the catheter system 400 may include a stiffener such as the stiffener 120 previously described. In some instances, a user can grip the stiffener 120 to advance the catheter 410 distally relative to the housing 411. In other or further embodiments, the catheter system 400 can include one or more positioning members, such as the positioning members 260, 262 discussed above with respect to FIG. 8. For example, the housing 411 can include a toothed track and the catheter 410 can include a ribbed follower that interfaces with the toothed track for controlled movement of the catheter relative to the housing 411, such as previously described.

In other or further embodiments, a distal end of the catheter 410 may extend past a distal end of the housing 411 when the catheter 410 is in a fully retracted orientation. In these and/or other embodiments, the catheter system 400 can include a cap, such as the cap 370 previously described.

FIG. 12 depicts another embodiment of a catheter system 500 that can resemble the catheter systems 100, 200, 300, 400 discussed above in many respects and, as previously described, features of these various systems may be incorporated into each other in any suitable manner. The catheter system 500 includes a catheter 510 that includes a proximal portion 514 and a distal portion 512. In some embodiments, the proximal and distal portions 514, 512 are joined at a junction 516. In other embodiments, the catheter 510 may have a unitary construction, such as previously disclosed. The catheter 510 can include a connector 534 at a proximal end of the proximal portion 514. In some embodiments, the catheter 510 can include a clamp 530 (or any other suitable fluid control device, such as previously discussed).

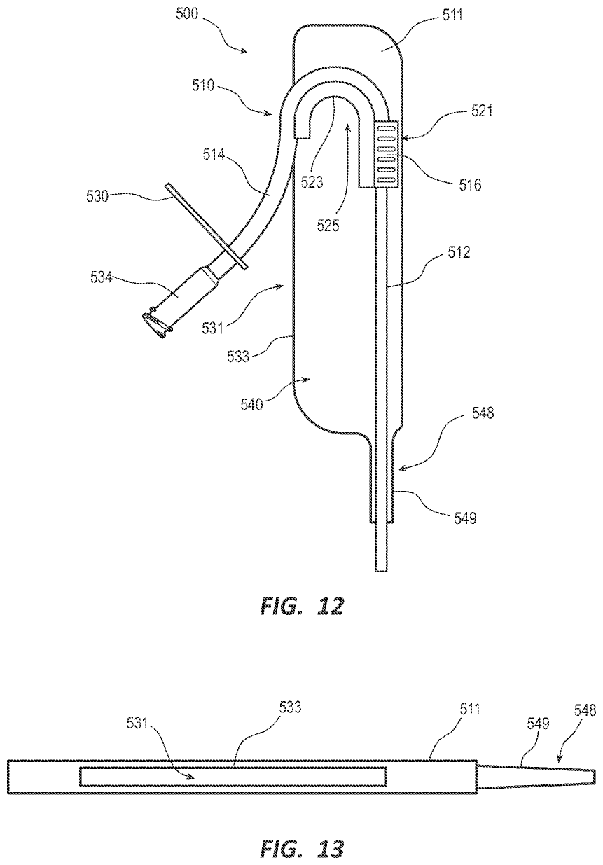

With reference to FIGS. 12 and 13, the catheter system 500 can further include a housing 511 relative to which the catheter 510 can be moved between retracted and deployed orientations. The housing 511 can define a chamber 540 within which a portion of the catheter 510 is received.

The housing 511 can define a relatively low profile in a first dimension and may define a relatively larger profile in a second dimension that is orthogonal to the first dimension, and the first and second dimensions may each be substantially orthogonal to an orientation of the distal portion 512 of the catheter 510. Stated otherwise, in some embodiments, the housing 511 may be elongated in a longitudinal direction, and can be wider than it is thick in transverse directions. The thickness may correspond to a low profile relative to a patient. The width can also facilitate grasping of the device by a user. In the illustrated embodiment, the housing 511 is substantially shaped as a rectangular cuboid or rectangular parallelepiped with rounded corners and edges. Other shapes are contemplated, and the housing 511 may be configured for enhanced ergonomics by which a user may comfortably hold the housing 511.

In certain embodiments, the housing 511 may define a shorter longitudinal length than may be achieved with similar arrangements of other embodiments disclosed herein, such as certain embodiments of the catheter systems 100, 200, 300, 400, for a fixed catheter length. For example, as further discussed below, the proximal portion 514 of the catheter 510 can be curved or bent so as to pass through an opening (e.g., an elongated opening) or track 531 in a sidewall 533 of the housing 511. The proximal portion 514, in some instances, may not be rigidly retained or maintained in a substantially rectilinear configuration that is aligned with a longitudinal axis of the distal portion 512 (such as the configuration depicted in the embodiment of FIG. 1). Stated otherwise, a central longitudinal axis of the proximal portion 514 may be curved or bent away from or otherwise deviate from a central longitudinal axis that extends through the distal portion 512. Such an arrangement can reduce an overall length of the catheter system 500, which may render the catheter system 500 more readily useable, steadier, and/or less unwieldy, which can result from having a relatively shorter moment arm and/or by permitting the hands of a user to be more closely situated relative to each other throughout use of the system 500.

The housing 511 can include an interface 548, which can include an elongate extension 549 such as similarly numbered items previously discussed. In the illustrated embodiment, the interface 548 is offset toward one side of the housing 511. Stated otherwise, a central longitudinal axis of the housing 511 does not pass through the interface 548, or stated otherwise, is offset from a central longitudinal axis that passes through the interface 548. The respective central longitudinal axes of the housing 511 and the interface 548 may be angled relative to one another or may be substantially parallel.

Similarly, a central longitudinal axis of the distal portion 512 of the catheter 510 may be offset from the central longitudinal axis of the housing 511, and further, may be aligned with the central longitudinal axis of the interface 548. During deployment, the distal portion 512 may be advanced distally along its own central longitudinal axis and that of the interface 548. The distal portion 512 may similarly be returned into the housing 511 proximally during retraction.

In some embodiments, the catheter 510 includes any suitable follower 521, such as any of the followers discussed above. The follower 521 and/or the housing 511 can include positioning members, such as any of those discussed previously herein, to assist with controlled deployment and retraction of the catheter 510.

In some embodiments, the catheter 510 includes a kink-prevention member 525 that supports a length of the proximal portion 514. In the illustrated embodiment, the kink-prevention member 525 comprises a curved support 523. The support 523 may be formed of a relatively rigid material and can maintain the proximal portion 514 in a patient state. The support 523 may be particularly useful, where the proximal portion 514 is curved so as to extend out of the sidewall 533 of the housing 511. In use, the proximal portion 514 may be gripped by the hand of a user and a force can be applied thereto to advance or retract the catheter 510 relative to the housing 511. The support 523 can prevent kinking of the proximal portion 514, or stated otherwise, can ensure that that catheter 510 remains patient after and/or during movement of the proximal portion 514, by which deployment and retraction of the catheter 510 is achieved. For example, a diameter of the support 523 can be greater than a diameter at which the proximal portion 514 kinks. In various embodiments, the diameter of the curved surface of the support 523 can be greater than the outer diameter of the tubular proximal portion 514 squared, divided by the thickness of the sidewall of the tubular proximal portion (e.g., [OD\hat{ }2]/t, where OD is the outer diameter and t is the thickness).

The support 523 may be fixedly secured to the proximal portion 514. For example, the support 523 may be overmolded onto the proximal portion 514. In other embodiments, the support 523 is molded separately and attached (e.g., via adhesive) to the proximal portion 514. Any suitable material is contemplated for the support 523, such as, for example, the materials discussed above with respect to embodiments of stiffeners.

In the illustrated embodiment, the proximal portion 514 projects laterally outwardly from the housing 511 with a distally directed component. Stated otherwise, the proximal member 514 is angled or directed forwardly. In some instances, this arrangement can facilitate gripping of the proximal portion 514 and/or the connector 534 and the controlled advancement and retraction of the catheter 510. For example, in some instances, a user can readily grip the proximal portion 514 and or the connector 534 with one or more finger of a hand, and can apply pressure with the thumb at or near the position at which the proximal portion 514 passes through the track 531. In advancing the catheter 510 in this configuration, it may be said that the user pulls the catheter 510 forward. Similarly, the user may be said to pull the catheter 510 backward during retraction.

In some embodiments, at least a portion of the proximal portion 514 of the catheter 510 can be covered or encompassed by a stiffener, such as embodiments of the stiffener 120 discussed above. For example, in some embodiments, the stiffener is separate from the support 523, and in further embodiments, the stiffener may be attached to the support 523 (e.g., via adhesive). In other embodiments, the stiffener and the support 523 may be integrally formed. The stiffener may extend out of the housing 511, positioned over at least a portion of the unhoused portion of the proximal portion 514 of the catheter 510.

In the illustrated embodiment, the catheter system 500 may readily or comfortably be used by either a right-handed person or a left-handed person, or may comfortably be used with either hand as a stabilizing hand that grips the housing 511 and the other hand as an activating hand that grips and moves or otherwise applies distally or proximally directed forces on the proximal portion 514 and/or the connector 534. For example, in one instance, a user may hold the housing 511 in the left hand with the proximal portion 514 projecting outwardly from the housing 511 toward the right, and may use the right hand to deploy and/or retract the catheter 510. In other instances, the housing 511 may be flipped over (e.g., rotated about the central longitudinal axis by approximately or exactly 180 degrees) such that the proximal portion 514 projects outwardly from the housing 511 toward the left. The user can grip the housing 511 in the right hand and may use the left hand to deploy and/or retract the catheter 510.

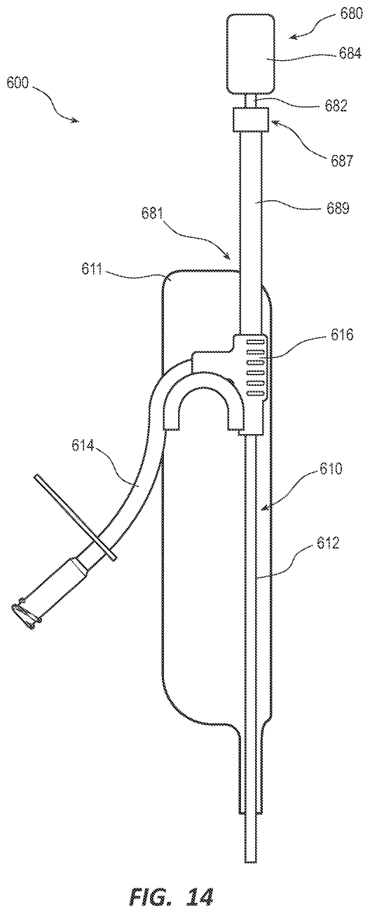

FIG. 14 depicts an embodiment of a catheter system 600 that can resemble the catheter systems 100, 200, 300, 400, 500 discussed above in many respects and, as previously described, features of these various systems may be incorporated into each other in any suitable manner. In particular, the catheter system 600 can substantially resemble the catheter system 500 described above, with the addition of an obturator assembly 680. The obturator assembly 680 can assist in moving a distal portion 612 of a catheter 610 through a vein. An obturator 682 can extend through a valve 687 and through a proximal tube 689, which may be relatively rigid to maintain a rectilinear form. The tube 689 can be coupled with a junction 616, via which the distal portion 612, the proximal portion 614, and the tube 689 are all joined and are internally in fluid communication with each other. The tube 689 can translate through an opening 681 in a housing 611 during advancement, retraction of the catheter 610.

As can be appreciated from the foregoing disclosure, in various embodiments, a catheter system can include a housing configured to couple with a base catheter that has a distal tip. The system can include a system catheter of which a portion is positioned within the housing and a proximal portion extends out of the housing, the system catheter being configured to move relative to the housing from a retracted position to an extended position. The system can further include a fitting coupled to the proximal portion of the system catheter. In certain embodiments, when the housing is coupled to the base catheter, the system catheter is advanced through the base catheter as the system catheter is moved to the extended position and, when the system catheter is in the extended position, a distal tip of the system catheter is positioned distally past the distal tip of the base catheter. In some instances, the system catheter is configured to be moved from the retracted position to the extended position by direct application of force to the proximal portion of the system catheter. In further instances, the system catheter is configured to be moved from the extended position to the retracted position by direct application of force to the proximal portion of the system catheter.

By way of example, a user can deploy or retract certain embodiments of the catheters 110, 210, 310, 410, 510, 610 by directly applying force (e.g., by gripping and/or pushing directly on) to one or more components of proximal portions of those catheters that extend out of the respective housings 111, 211, 311, 411, 511, 611. By way of further example, a user can deploy or retract certain embodiments of the aforementioned catheters by directly contacting and applying force to an uncovered or unexposed proximal portion (e.g., 114, 514, 614, as labeled in the drawings) of the respective catheters. With respect to embodiments of the catheter system 500, for example, a user may push the proximal portion 514 distally to deploy the catheter 510, and may pull proximally on the proximal portion 514 to retract the catheter 510.

As previously discussed, in various embodiments, a first catheter can be introduced into a patient, and subsequently, a second catheter can be introduced into the patient through the first catheter. The first catheter may be referred to herein as a placed catheter, an anchor catheter, or a base catheter, and the second catheter may be referred to herein as a follow-on catheter. In certain instances, when the follow-on catheter is coupled to the base catheter, there may be an open space between the base and follow-on catheters through which blood can leak from the patient. In some instances, the open space, which may also be referred to herein as an egress path or passageway, may include an annular region that extends longitudinally between an outer surface of the follow-on catheter and an inner surface of the base catheter. In other or further instances, the egress passageway may include an annulus (e.g., a conical annulus) that extends longitudinally between respective connectors (e.g., fluid connection fittings) positioned at proximal ends of the follow-on and base catheters. Various embodiments herein are configured to occlude or seal the egress passageway to prevent blood from leaking out from the catheter hubs.

For example, in some embodiments, a follow-on catheter includes a tube connected to a hub connection, such as a luer or a threaded luer. The tube can be constructed of a thin strong material (e.g., PEEK), being layered with a sealing member, such as a swelling material (e.g., a hydrogel) that swells due to contact with aqueous liquids. The swelling layer, such as a coating or thin tube sleeve, can have a property that allows sufficient swelling to occlude the annular space between respective catheter tubes when the follow-on catheter is inserted into the lumen of the base catheter, when the coupled system is in contact with an aqueous solution (e.g., blood). The swelling layer can exhibit high lubricity or low shear resistance in order to permit or facilitate removal of the follow-on catheter from the inner lumen of the base catheter.

In other or further embodiments, a follow-on catheter can include a tube connected to a hub connection, such as a luer or a threaded luer. The tube can be constructed of a thin strong material (e.g., PEEK). The hub can include a sealing member that provides a seal with the hub of the base catheter. For example, in some embodiments, the follow-on hub can include a swelling layer at an exterior thereof that swells when in contact with an aqueous solution (e.g., blood) to seal or occlude an open annular space (e.g., a conical annulus) between an outer surface of the follow-on hub and an inner surface of the base hub. In other or further embodiments, the follow-on hub can include a seal element, such as an elastomeric O-ring, at an exterior thereof that seals the annular egress passageway between the follow-on and base hubs. In still other or further embodiments, at least a portion of the outer surface of the follow-on hub includes a male luer configuration that is configured to form a frictional seal with an internal female luer configuration of the base hub, thereby preventing any blood leakage between the follow-on and base catheter systems.

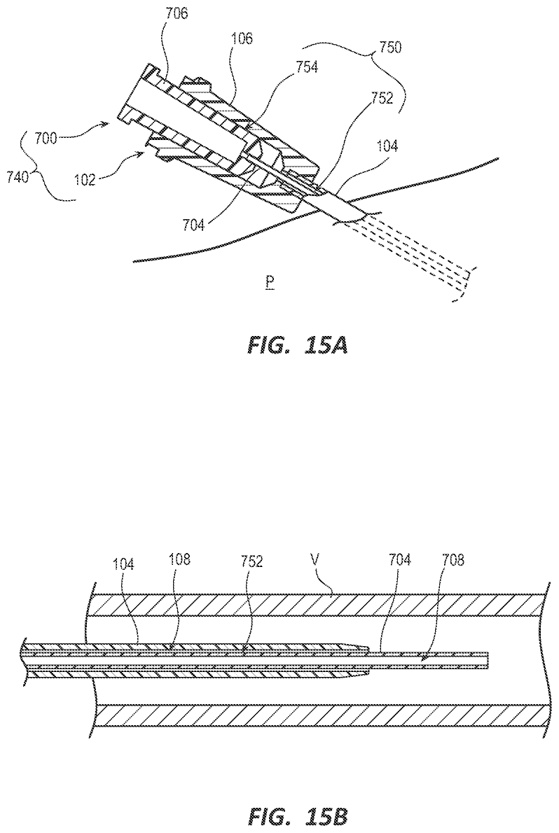

FIGS. 15A and 15B depict an embodiment of a base catheter system 102, such as previously described. The base catheter system 102 includes a catheter tube 104 and a hub 106 that is fixedly secured to a proximal end of the catheter tube 104. The base catheter system 102 can be inserted into a patient such that at least a distal end of the tube 104 extends into a vessel V (e.g., a vein) of the patient (FIG. 15B) and such that a proximal portion of the catheter system 102, including the hub 106, is accessible at an exterior of the patient. The tube 104 can define a lumen 108 through which, for example, infusions may be delivered into the vessel V. The hub 106 may be of any suitable variety. In some embodiments, the hub 106 is a female luer connector.

With continued reference to FIGS. 15A and 15B, in some embodiments, a follow-on catheter 700 can be configured to couple with the base catheter system 102. The follow-on catheter 700 can include a catheter tube 704 of which a portion can be inserted through the base catheter tube 104. The catheter tubes may also be referred to as catheter bodies.

The follow-on catheter 700 can further include a hub 706 that is fixedly secured (e.g., bonded) to a proximal end of the catheter tube 704. The hub 706 may for formed as or otherwise include a connector that is configured to couple with any suitable fluid source and/or fluid collection device or may, in other embodiments, be integrally formed with fluid source and/or fluid collection apparatus. For example, in the illustrated embodiment, the hub 706 comprises a proximal end that is shaped as a female luer connector configured to couple with a syringe, which may be used for infusion or blood collection. The illustrated hub 706 and/or other varieties of connectors may be used for connection to blood draw apparatus, such as evacuated blood collection tubes (e.g., Vacutainer® tubes available from Becton Dickinson) and/or apparatus therefor. For example, in some embodiments, the hub 706 may include or may be configured to connect with a Vacutainer® one-use holder, via which blood draws may be made into one or more Vacutainer® tubes.

In the illustrated embodiment, the hub 706 does not provide any valving relative to a lumen 708 that extends through the catheter tube 704. In some instances, a clamp (not shown) may selectively permit fluid flow through the catheter tube 704. In other embodiments, the hub 706 may comprise a valve and/or a clamp may be omitted. For example, in some embodiments, the hub 706 comprises a needleless connector valve of any suitable variety.

In some embodiments, as further discussed below, the hub 706 may be configured to securely couple with the hub 106 so as to form a seal therewith. For example, in some embodiments, the hub 106 may define a luer interface. For example, the hub 106 may be formed as a female luer connector or as a female luer lock connector. In some embodiments, hub 706, or at least a distal end thereof, can be shaped as a male luer for insertion into and frictional engagement with the hub 106. In other embodiments, the hub 706 may comprise a luer lock connection, such that the hub 706, or a portion thereof, may be rotated relative to the hub 106 to securely connect the hubs 106, 706 to each other, and may be rotated again after use for disconnection. Any other suitable interfacing and/or locking arrangement between the systems hubs 106, 706 is contemplated.

The catheter tube 704 can be inserted through the base hub 106 and advanced distally through the base catheter tube 104 such that a distal tip of the follow-on catheter tube 704 extends beyond a distal tip of the base catheter tube 104 and into the bloodstream of the vessel V. Fluid flow into or from the vessel V—namely, infusions or aspirations, and blood draws in particular—can be performed through a lumen 708 defined by the catheter tube 704. The follow-on catheter 700 may be said to bypass or supersede the base catheter 100 and can be used for functions that might otherwise have been performed directly via the base catheter 100, such as infusions and/or blood collections. Accordingly, the follow-on catheter 700 may also be referred to herein as a bypass, superseding, add-on, or transient catheter, whereas the base catheter 100 may alternatively be referred to as a pre-placed, placed, or anchor catheter. In some instances, the base and follow-on catheters 102, 700 may be referred to as a catheter system 740.

In certain instances, when the follow-on catheter 700 is coupled to the base catheter 102, there may be an open space or egress passageway 750 between the base and follow-on catheters 102, 700 through which blood can leak from the patient P. Stated otherwise, the catheter system 740, when in a coupled configuration, may define the egress passageway 750 between the base and follow-on catheters 102, 700. In some instances, the egress passageway 750 may include an annular region 752 that extends longitudinally between an outer surface of the follow-on catheter tube 104 and an inner surface of the base catheter tube 704. Stated otherwise, the annular region 752 may be defined as the portion of the lumen 108 of the catheter tube 104 that is external to the catheter tube 704. In other or further instances, the egress passageway may include further annular region 754 (e.g., a conical annulus) that extends longitudinally between the connectors or hubs 106, 706. Various embodiments discussed hereafter are configured to occlude or seal the egress passageway to prevent blood from leaking from the coupled catheters 102, 700.

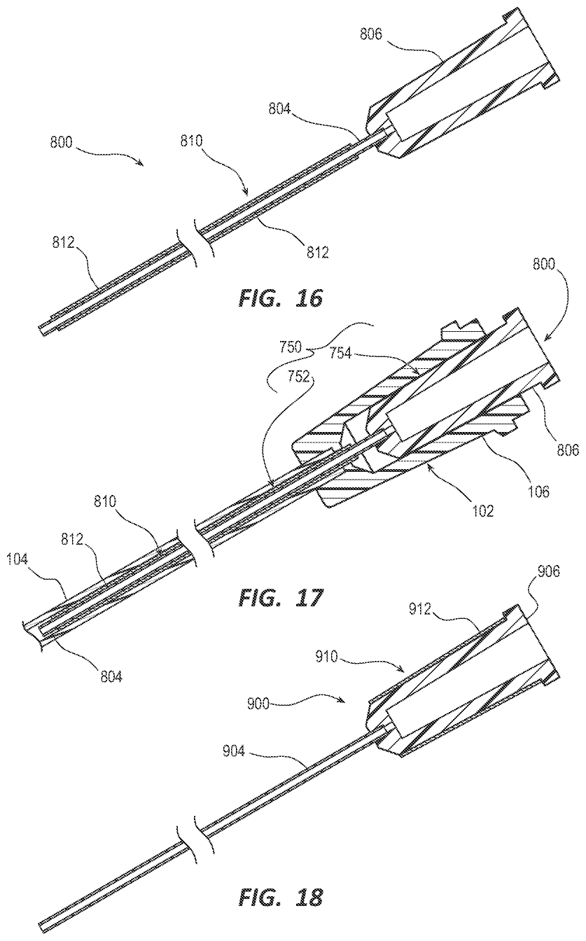

FIG. 16 depicts an embodiment of a follow-on catheter 800, which can resemble the follow-on catheter 700 discussed above in many respects and may be used with embodiments of the base catheter 102. The illustrated catheter 800 includes a catheter tube 804 and a hub 806. The hub 806 can be affixed to a proximal end of the catheter tube 804 in any suitable manner, such as, for example, via overmolding or bonding. In some embodiments, at least a portion of the catheter tube 804 includes a sealing member 810. In the illustrated embodiment, the sealing member 810 is formed as a swelling layer 812 that is fixedly secured to the catheter tube 804 at an external surface thereof. The swelling layer 812 is positioned so as to come into contact with an aqueous solution (e.g., blood) that otherwise would move past an exterior of the catheter tube 804.

In some embodiments, the swelling layer 812 is coated onto the outside surface of the tube 804. In other or further embodiments, the swelling layer 812 comprises a sleeve positioned at an outer surface of the tube 804. For example, in some embodiments, the swelling layer 812 comprises a sleeve of material that is bonded to an outside surface of the tube 804. In still other or further embodiments, the sealing member 810 may be incorporated into the body of the catheter tube 804. For example, in some embodiments, the sealing member 810 may be extruded as, or coextruded with, the material that defines the inner lumen of the catheter tube 804. In some instances, it may be advantageous for the swelling layer 812 to be positioned only at an exterior of the catheter tube 804, as this may assist in maintaining the lumen of the tube 804 patient.

The swelling layer 812 can fully encompass at least a portion of the catheter tube 804. For example, the swelling layer 812 may fully encircle the catheter tube 804 along at least a portion of a length of the catheter tube 804. In some instances, fully encompassing the catheter tube 804 can ensure a tight seal is formed to prevent blood from leaking from the system.

In the illustrated embodiment, the swelling layer 812 extends along substantially a full length of the tube 804. In other embodiments, the swelling layer 812 may extend along only a portion of the tube length, such as, for example, no greater than 1/30, 1/20, 1/10, ⅕, ¼, ⅓, or ½ of the length of the tube 804. In various embodiments, the swelling layer 812 is at only a distal, intermediate, or proximal region of the tube 804. In other or further embodiments, and as further discussed below, in some embodiments, the swelling layer 812 can be included at an external surface of at least a portion of the hub 806, whether instead of or in addition to the outer surface of the tube 804.

The swelling layer 812 may be formed of any suitable material capable of swelling or expanding when in contact with or exposed to an aqueous solution, such as blood. For example, in various embodiments, the material can comprise a hydrogel. In some embodiments, the material is hydrophilic. In other or further embodiments, the material is hydrophobic. In some instances, a portion of the swelling layer 812 includes a hydrophilic material, while a different portion of the swelling layer 812 includes a hydrophobic material.

In some embodiments, the tube 804 may be a thin-walled tube. For example, a relatively thin wall may permit relatively rapid and/or relatively high quality (e.g., with little or no hemolysis) blood draws, while still permitting insertion of the tube 804 through the lumen of the tube 104 of the base catheter 102. In various embodiments, a total thickness of the wall of the tube 804, including the swelling layer 812, is no greater than 0.001, 0.003, 0.005, or 0.010 inches. In certain of these and/or other embodiments, the swelling layer 812 can be configured to expand to a thickness of between 0.001 and 0.030 inches.

With reference to FIG. 17, illustrative examples of using embodiments of the base catheter 102 with embodiments of the follow-on catheter 800 will now be described. In certain embodiments, the base catheter 102 is pre-placed within the vein of a patient according to accepted protocols. The base catheter 102, for example, may be a peripherally intravenous (PIV) catheter.

The follow-on catheter 800 can be inserted through the hub 106 and advanced until the distal tip of the catheter tube 804 extends past a distal tip of the base catheter tube 104. In various instances, the distal tip of the catheter tube 804 is advanced past the distal tip of the base catheter tube 104 by a sufficient distance and/or to otherwise achieve an orientation in which one or more exposed openings at a distal end of the catheter tube 804 are unobstructed by clots or fibrin sheaths, is extended past one or more venous valves, is positioned away from a vessel wall, and/or is positioned within a region of increased blood flow, any or all of which can result in substantially improved blood draws. In the illustrated embodiment, the opening is a simple circular and planar opening oriented transversely relative to a longitudinal axis of the catheter tube 804 and positioned at the distal tip of the catheter tube 804. Any other suitable variety of opening configuration is contemplated. In other instances, the distal tip of the catheter tube 804 may be positioned flush with the distal tip of the catheter tube 104 or may be recessed proximally from the distal tip of the catheter tube 104.

With the catheter tube 804 in a desired position within the catheter tube 104 and vein, blood may naturally pass through the annular region 752 of the egress passageway 750 (see also FIGS. 15A and 15B). When the blood comes in contact with the swelling layer 812, the swelling layer can expand. The expansion can be sufficient to occlude or seal the annular region 752, thereby closing the egress passageway 750 and preventing blood from inadvertently exiting the coupled catheters 102, 800 through a region that is between the catheters 102, 800, or stated otherwise, along a path that is external to the hub 806.

In the case of blood collection, a blood collection device, such as any of those described above, can be coupled to the hub 806. In the case of infusion, an infusion device, such as any of those described above, can be coupled to the hub 806. After the withdrawal or delivery of fluid via the follow-on catheter 800, the follow-on catheter 800 may be removed from the base catheter 102 and disposed of appropriately. In some embodiments, the swelling layer 812 is able to be readily retracted through the catheter tube 104 of the base catheter 102 without disrupting the placement or positioning of the catheter tube 104. The base catheter 100 may remain in place within the patient after use of the follow-on catheter 800 therewith.

FIG. 18 depicts an embodiment of a follow-on catheter 900 that includes a sealing member 910 on a hub 906. In particular, in the illustrated embodiment, the hub 906 includes a swelling layer 912 similar to the swelling layers previously discussed. The swelling layer 912 may fully encompass at least a portion of the hub 906.

In the illustrated embodiment, the swelling layer 912 is positioned exclusively on the hub 906. In other embodiments, a catheter tube 904 may also include a swelling layer.

In operation, the follow-on catheter 900 may be inserted the base catheter 102, which may have previously been placed within the patient. Blood may pass from the vein proximally through the annular region 752 (see FIGS. 15A, 15B, and 17) until it reaches the swelling layer 912. The blood can cause the swelling layer 912 to expand and seal off the annular region 754 (see FIGS. 15A, 15B, and 17), thus, more generally, sealing the egress passageway 750.

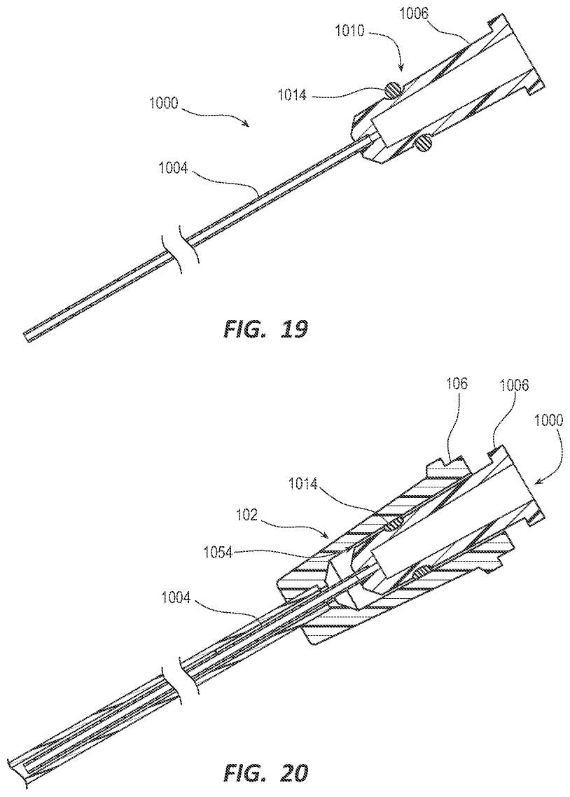

FIGS. 19 and 20 depict an embodiment of a follow-on catheter 1000 that includes a sealing member 1010 on a hub 1006. In particular, in the illustrated embodiment, the hub 1006 includes a seal element 1014 that is configured to seal the annular region 754 between the hubs 106, 1006 of the catheters 100, 1000. In some embodiments, the seal element 1014 is configured to be deformed (e.g., resiliently deformed), such as compressed, as the hub 1006 is coupled with the hub 106. In still other or further embodiments, the seal element 1014 (or an additional seal element 1014) is positioned at an exterior surface of the tube 1004.

One or more seal elements 1014 can be, for example, one or more raised rings on the outside surface of the catheter 1000—e.g., an outside surface of the tube 1004 and/or the hub 1006. In some embodiments, a sealing ring can be positioned at an exterior of the hub 1006 and can have dimensional characteristics to seal on the inside surface of a female luer hub 106 of a base catheter 102. In some embodiments, seal element 1014 is a separate ring (e.g., an O-ring or the like) that is attached to the outside surface of the hub 1006 or the tube 1004. In other or further embodiments, one or more seal elements 1014, such as raised rings, are integrally molded with one or more of the hub 1006 or the tube 1004.

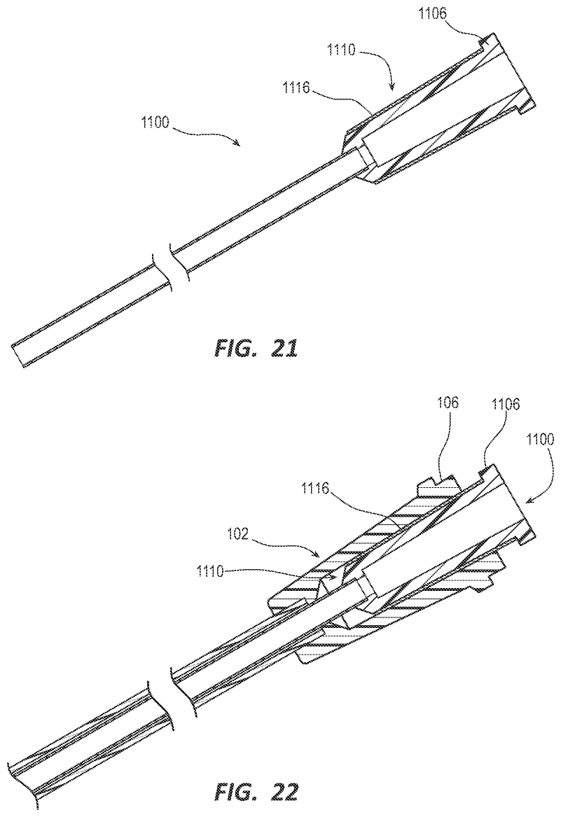

FIGS. 21 and 22 depict an embodiment of a follow-on catheter 1100 that includes a sealing member 1110 on a hub 1106. In particular, in the illustrated embodiment, the hub 1106 includes an external male luer interface 1116 at a distal end thereof that is configured to frictionally engage and seal with a female luer interface 118 of the hub 106. The seal thus formed can close the annular region 754 to prevent the egress of blood. In the illustrated embodiment, the hub 1106 further includes a female luer 1118 configuration, or female luer connector, at a proximal end thereof. The female luer 1118 is configured to couple with fluid delivery and/or fluid collection devices of any suitable variety. The hub 1106 may be described as a female luer connector that includes a male luer connector for coupling the hub 1106 to the hub of another catheter. The female luer connector can be at the proximal end of the hub 1106, and the male luer connector can be at the distal end of the hub 1106.

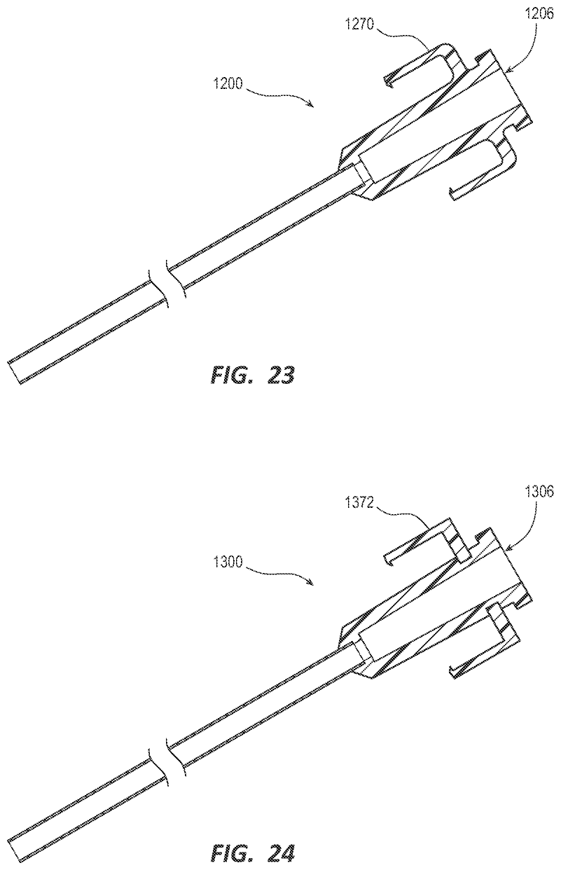

FIG. 23 depicts an embodiment of a follow-on catheter 1200 that varies from the catheter 1100 in only one way. In particular, the follow-on catheter 1200 further includes a luer lock connector 1270 by which a hub 1206 of the catheter 1200 can be secured to a hub 106 of the base catheter 102 that includes a complementary luer lock connector. In the illustrated embodiment, the luer lock connector 1270 is integral to e.g., integrally molded with) and fixed relative to the remainder of the hub 1206. To connect the hubs 1206, 106, a user rotates one or more of the hubs 1206, 106 relative to the other. This can result in rotation of the catheter tubes relative to each other.

FIG. 24 depicts an embodiment of a follow-on catheter 1300 that varies from the catheter 1200 in only one way. In particular, the follow-on catheter 1300 includes a luer lock connector 1372 that is rotational relative to the remainder of a hub 1306. In particular, the luer lock connector 1372 may be axially constrained, or stated otherwise, longitudinal movement of the luer lock connector 1372 can be delimited. The luer lock connector 1372 may further be freely rotational, or stated otherwise, may be rotationally unconstrained. To connect the hubs 1306, 106, a user rotates the luer lock connector 1372 relative to the hub 106 to tighten the hubs 1306, 106 together. The hubs 1306,106 and the catheter tubes attached thereto can remain rotationally fixed relative to each other during connection and disconnection of the luer lock connector 1372, as the luer lock connector 1372 is free to rotate independent of the hub 1306.

The term “coupled to” can mean connected to in any suitable fashion, whether that coupling is direct or indirect. Separate components may be coupled to each other. Moreover, in some instances, where separately identified components are integrally formed from a unitary piece of material, or stated otherwise, are included together in a monolithic element, those elements may also be said to be coupled to one another.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of the preceding claims up to and including claim [x],” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Embodiments of the invention in which an exclusive property or privilege is claimed are defined as follows.

1. A system comprising:
a housing configured to be coupled with a base catheter, the housing defining an opening;
an access catheter of which a first portion is positioned within the housing and a second portion extends out of the housing through the opening when the access catheter is in a retracted position, the access catheter being configured to move relative to the housing from the retracted position to an extended position;
a stiffener encompassing at least a portion of the second portion of the access catheter, the stiffener being graspable at an exterior of the housing for application of force thereto to advance the access catheter from the retracted position to the extended position; and
a fitting coupled to the second portion of the access catheter,
wherein when the housing is coupled to the base catheter, the first portion of the access catheter is advanced through at least a portion of the base catheter as the access catheter is advanced to the extended position.

2. The system of claim 1, wherein the access catheter is configured to be returned from the extended position to the retracted position by direct application of force to the stiffener.

3. The system of claim 1, wherein the stiffener encompasses no less than 50 percent of a portion of the access catheter that is external to the housing when the access catheter is in the retracted position.

4. The system of claim 1, wherein the stiffener is overmolded onto the access catheter.

5. The system of claim 1, wherein a hardness of the stiffener is greater than a hardness of the second portion of the access catheter.

6. The system of claim 1, wherein the first portion of the access catheter is softer than the second portion of the access catheter.

7. The system of claim 1, further comprising a junction that joins the first and second portions of the catheter together.

8. The system of claim 7, wherein the stiffener is attached to the junction.

9. The system of claim 8, wherein the stiffener and the junction are overmolded onto the access catheter.

10. The system of claim 1, further comprising a follower positioned within the housing and coupled to the access catheter, the follower being constrained to remain within the housing to prevent removal of the access catheter from the housing.

13. The system of claim 1, wherein the opening defined by the housing is at a proximal end of the housing, and wherein the access catheter is advanced through a distal end of the housing.

14. The system of claim 1, wherein the opening defined by the housing comprises a track that extends longitudinally at a side of the housing.

15. The system of claim 1, further comprising an obturator assembly coupled to the assembly catheter, wherein the obturator assembly comprises a removable obturator that extends through at least the first portion of the access catheter.

16. The system of claim 15, wherein the obturator extends through the second portion of the access catheter.

17. The system of claim 16, wherein the obturator assembly further comprises a valve through which the obturator extends.

18. The system of claim 17, wherein the obturator extends through the opening defined by the housing.

19. The system of claim 17, wherein the housing defines a separate additional opening, and wherein the obturator extends through the additional opening of the housing.

20. The system of claim 1, wherein the second portion of the access catheter comprises a curved region, and wherein the system further comprises a support coupled to the curved region.
